(19) United States
(12) Patent Application Publication
(10) Pub. No.: US 2022/0208381 A1
(43) Pub. Date: Jun. 30, 2022

(54) MEDICAL CARE SUPPORT DEVICE, OPERATION METHOD AND OPERATION PROGRAM THEREOF, AND MEDICAL CARE SUPPORT SYSTEM

(71) Applicant: FUJIFILM Corporation, Tokyo (JP)

(72) Inventors: Tsuyoshi HIRAKAWA, Tokyo (JP); Hiroshi HIRAMATSU, Tokyo (JP); Keiji TSUBOTA, Tokyo (JP); Misaki KAWAHARA, Tokyo (JP)

(73) Assignee: FUJIFILM Corporation, Tokyo (JP)

(21) Appl. No.: 17/695,867

(22) Filed: Mar. 16, 2022

Related U.S. Application Data
(63) Continuation of application No. PCT/JP2020/030784, filed on Aug. 13, 2020.

(30) Foreign Application Priority Data
Sep. 27, 2019 (JP) 2019-177805

(51) Int. Cl. G16H 50/20

(52) U.S. Cl. CPC G16H 50/20

(57) ABSTRACT

A medical care support device (11) includes an operation history acquisition unit (63), a prediction execution unit (64), and a display screen generation unit (62). The operation history acquisition unit (63) acquires an operation history in a case where a terminal device is operated. The prediction execution unit (64) predicts a next operation candidate in a case where the terminal device is input and operated by using a trained model generated by an external server learning the acquired operation history, the external server being installed outside the medical facility. The display screen generation unit (62) makes a proposal to the terminal device from the next operation candidate predicted by the prediction execution unit (64).

### CROSS-REFERENCE TO RELATED APPLICATIONS

This application is a Continuation of PCT International Application No. PCT/JP2020/030784 filed on Aug. 13, 2020, which claims priority under 35 U.S.C § 119(a) to Japanese Patent Application No. 2019-177805 filed on Sep. 27, 2019. Each of the above application(s) is hereby expressly incorporated by reference, in its entirety, into the present application.

### BACKGROUND OF THE INVENTION

### 1. Field of the Invention

The present invention relates to a medical care support device, an operation method and non-transitory computer readable recording medium storing an operation program thereof, and a medical care support system.

### 2. Description of the Related Art

In the medical field, integrated medical care support devices and medical care support systems that share medical care processes and medical care results between medical staff or medical departments so that the medical staff such as doctors and laboratory technicians can smoothly proceed with medical examinations and tests are being used. The medical care support device supports medical care by providing the medical staff with, for example, displaying a list of medical care processes and medical care results for a plurality of patients (JP2016-143204A).

On the other hand, in the medical field as well, the work of medical staff is streamlined by using machine learning, and for example, in an information processing apparatus described in JP5151913B, an operation history performed at the time of the past medical examination is analyzed and learned on an operation screen of an electronic medical record or the like. Then, for the medication or disease name input and operated by the medical staff, the next operation is predicted based on the learning result. The predicted operation is proposed as the next operation candidate.

### SUMMARY OF THE INVENTION

Data including personal information of patients is handled in such a manner that the medical care support device and medical care support system described in JP2016-143204A display medical care information and medical care results, and the information processing apparatus described in JP5151913B performs analysis and learning from an operation screen of an electronic medical record. Therefore, in order to avoid a risk of leakage of personal information or the like of patients, each hospital facility is often operated only by the network inside the facility.

Further, in a case where the medical care support device and the medical care support system as described in JP2016-143204A are allowed to analyze and learn the operation history as in the information processing apparatus described in JP5151913B, the following problems occur. For example, in machine learning about a device that recognizes a lesion arear or the like with respect to a medical image, many medical images can be accumulated and learned in advance. However, in a case where the operation history is learned in the medical care support device and the medical care support system, it is necessary to accumulate and learn the operation history in the case of being operated by the same device, the same system, and at least the medical staff of the same job type as the medical care support device and medical care support system used in a predetermined hospital facility. That is, in the case of learning results generated based on operation histories in which even any one of the device, the system, and the job type is different, it is difficult to obtain a high learning effect.

Further, in machine learning, prediction accuracy can be improved by accumulating more samples and continuing learning appropriately, but considering the leakage of personal information or the like of patients, it is not possible to collect operation histories related to a large number of various users only by learning with a medical care support device and a medical care support system in one hospital facility, and thus it is not possible to improve the prediction accuracy.

Therefore, an object of the present invention is to provide a medical care support device, an operation method and a non-transitory computer readable recording medium storing an operation program thereof, and a medical care support system capable of collecting operation histories of many users and improving prediction accuracy while avoiding the risk of leakage of patient information.

According to an aspect of the present invention, there is provided a medical care support device comprising an operation history acquisition unit, a prediction execution unit, and an operation proposal unit. The operation history acquisition unit acquires an operation history in a case where a terminal device installed in a medical facility is operated, from the terminal device. The prediction execution unit predicts a next operation candidate in a case where the terminal device is input and operated by using a trained model generated by an external server learning the acquired operation history, the external server being installed outside the medical facility. The operation proposal unit makes a proposal to the terminal device from the next operation candidate predicted by the prediction execution unit.

It is preferable that user identification information for specifying the user who uses the terminal device is attached to the operation history.

It is preferable that the trained model is generated by the external server learning the operation history, and in a case where personal information is included in the operation history and the operation history is transmitted to the external server, the personal information is deleted.

It is preferable that the trained model is generated by the external server learning the operation history, and in a case where personal information is included in the operation history and the operation history is transmitted to the external server, the personal information is deleted, and a portion of the personal information part in the operation history is accumulated in an internal storage device installed in the same medical facility as the terminal device.

It is preferable that the operation history includes at least examination data, as an operation target.

It is preferable that the operation history includes at least one of an order of examination data referred to by the user, a change of a display layout input and operated by the user, or a display magnification, as an operation target.

It is preferable that the operation history includes at least one of a type of created document created by the user, an order in which the created document is created, or a creation time of the created document, as an operation target.

It is preferable that the operation history includes at least one of a function used by the user or an order in which the function is used, as an operation target.

It is preferable that the operation history includes at least a type of examination or treatment ordered by the user through the terminal device.

It is preferable that the operation history includes at least an order time indicating a time point or a time slot at which the user ordered an examination or treatment through the terminal device.

It is preferable that the operation proposal unit displays examination data on the terminal device as the proposal.

It is preferable that the operation proposal unit changes a layout of examination data displayed on the terminal device as the proposal.

It is preferable that the operation proposal unit performs, as the proposal, a display of a created document to be created by the user, or a display prompting creation of the created document by using the terminal device.

It is preferable that the operation proposal unit displays, as the proposal, an operation content to be input and operated by the user by using the terminal device.

It is preferable that the operation proposal unit performs, as the proposal, a display of an examination or treatment to be ordered by the user, or a display prompting the user to order by using the terminal device.

It is preferable that the operation proposal unit makes the proposal according to a time point or a time slot.

According to another aspect of the present invention, there is provided a medical care support system comprising a medical care support device, a terminal device, and an external server.

According to another aspect of the present invention, there is provided an operation method of a medical care support device, the operation method comprising: an operation history acquisition step of acquiring an operation history in a case where a terminal device installed in a medical facility is operated, from the terminal device; a prediction execution step of predicting a next operation candidate in a case where the terminal device is input and operated by using a trained model generated by an external server learning the acquired operation history, the external server being installed outside the medical facility; and an operation proposal step of making a proposal to the terminal device from the predicted next operation candidate.

According to another aspect of the present invention, there is provided a non-transitory computer readable recording medium storing an operation program of a medical care support device, the operation program comprising: an operation history acquisition step of acquiring an operation history in a case where a terminal device installed in a medical facility is operated, from the terminal device; a prediction execution step of predicting a next operation candidate in a case where the terminal device is input and operated by using a trained model generated by an external server learning the acquired operation history, the external server being installed outside the medical facility; and an operation proposal step of making a proposal to the terminal device from the predicted next operation candidate.

According to the aspects of the present invention, it is possible to collect the operation histories of many users and improve the prediction accuracy while avoiding the risk of leakage of patient information.

### BRIEF DESCRIPTION OF THE DRAWINGS

FIG. 1 is an explanatory diagram showing a configuration of a medical care support system.

FIG. 2 is an explanatory diagram showing a configuration of a network provided in a medical facility.

FIG. 3 is a block diagram showing a configuration of a client terminal.

FIG. 4 is a block diagram showing a function of the client terminal.

FIG. 5 is a block diagram showing a configuration of a medical care support device.

FIG. 6 is a block diagram showing a function of the medical care support device.

FIG. 7 is an explanatory diagram showing an example of an operation history.

FIG. 8 is a block diagram showing a function of a learning device.

FIG. 9 is an initial screen.

FIG. 10 is a layout display screen.

FIG. 11 is a layout display screen on which the next operation is proposed.

FIG. 12 is a layout display screen showing a modification example of a first embodiment.

FIG. 13 is an explanatory diagram showing an example of an operation history in a second embodiment.

FIG. 14 is an explanatory diagram showing an example of an operation history in a third embodiment.

FIG. 15 is an explanatory diagram showing an example of an operation history in a fourth embodiment.

FIG. 16 is an example of a layout display screen in the fourth embodiment.

FIG. 17 is an example of an operation history in a fifth embodiment.

FIG. 18 is an explanatory diagram showing an example in which personal information is deleted from an operation history.

FIG. 19 is an explanatory diagram showing an example in which personal information deleted from an operation history is accumulated in an internal storage device.

### DESCRIPTION OF THE PREFERRED EMBODIMENTS

### First Embodiment

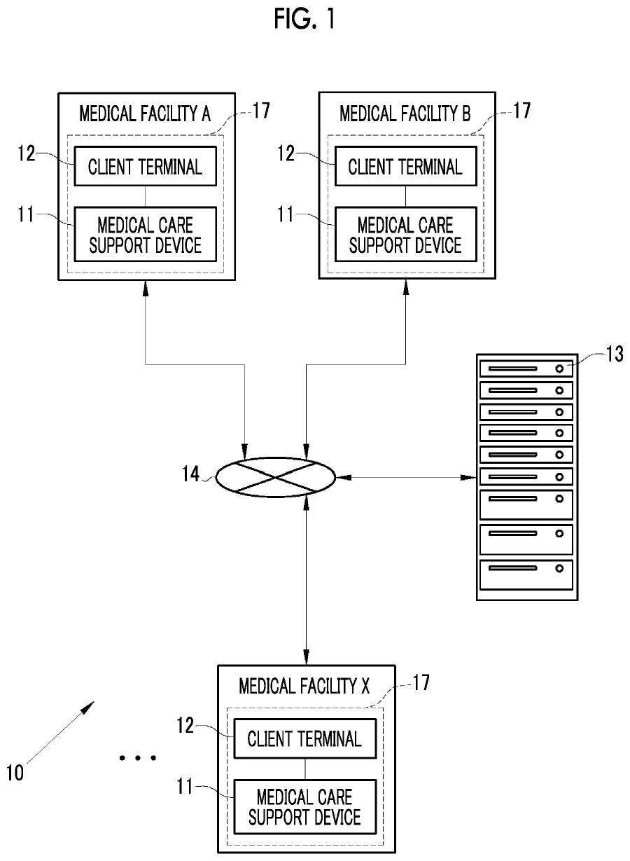

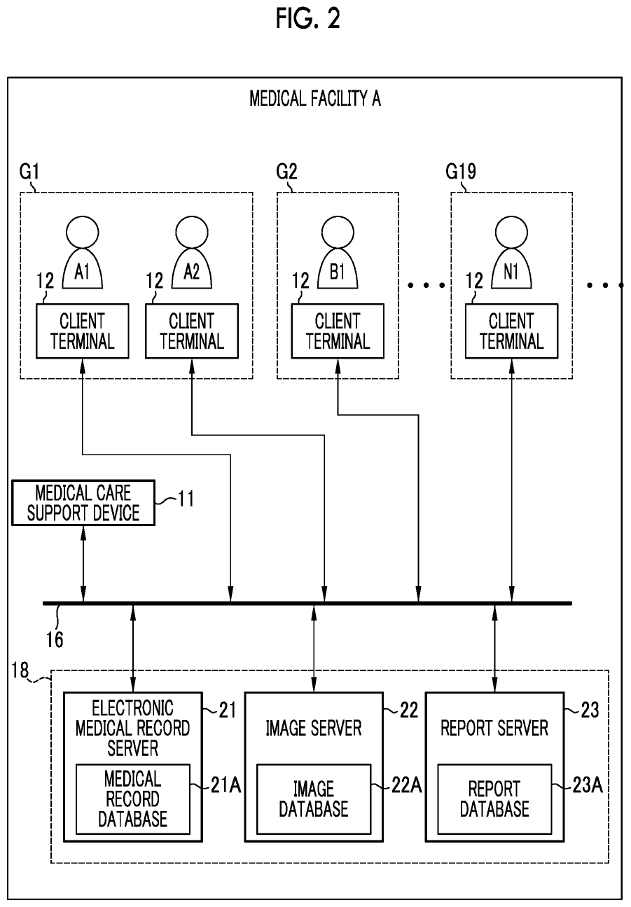

As shown in FIG. 1, a medical care support system 10 is a computer system that provides medical care support in a medical facility such as a hospital, and comprises medical care support devices 11 installed in a plurality of medical facilities A, B, . . . , X, client terminals 12 installed in the same medical facilities A, B, . . . , X as the medical care support device 11, a learning device 13, a network 14, and the like. The medical care support system 10 includes a medical information system 17 (see FIG. 2) provided in each of the medical facilities A, B, . . . , X. Further, a plurality of medical care support devices 11 may be installed in each of the medical facilities A, B, . . . , X. The learning device 13 is an external server installed on the cloud.

The network 14 is a wide area network (WAN) that widely connects the medical care support device 11 placed in the plurality of medical facilities A, B, . . . , X and the learning device 13 via a public line network such as the Internet or a dedicated line network.

As shown in FIG. 2, the medical care support device 11 is connected to the medical information system 17 provided in the medical facility A via the network 16 installed in one medical facility A. Although not shown, the medical information system 17 is similarly provided in the other medical facilities B, . . . , X, and the medical care support device 11, the client terminal 12, and the like are connected to the network 16 as in FIG. 2.

The medical information system 17 comprises the medical care support device 11, the client terminal 12, and a server group 18, and is configured to be able to transmit and receive data to and from each other via a network 16. The network 16 is a local area network (LAN), and it is desirable to use a communication cable such as an optical fiber so that medical image data can be transmitted at high speed.

The client terminal 12 (terminal device) is a terminal for receiving a service (a function of the medical care support device 11) from the medical care support device 11, and is a computer directly operated by a medical staff such as a doctor, a laboratory technician, or a nurse (including the case of a tablet terminal, etc.), or the like. The client terminal 12 is installed in a medical department such as an internal medicine or a surgery, various examination departments such as a radiological examination department or a clinical examination department, a nurse center, or other necessary places. Further, the client terminal 12 can be provided for each medical staff, and can be shared by a plurality of medical staff. Therefore, as shown in FIG. 2, the medical information system 17 includes a plurality of client terminals 12. For example, a group G1 is an “internal medicine” to which a doctor A1 and a doctor A2 belong, and the doctor A1 and the doctor A2 each have a client terminal 12. Similarly, for example, a group G2 is a “surgery” to which a doctor B1 belongs, and the group G2 has at least one client terminal 12. Further, for example, a group G19 is a “radiology department” to which a technician N1 belongs, and the group G19 has at least one client terminal 12.

The medical care support device 11 provides the client terminal 12 with a display screen including medical care data (for example, an image or the like itself) and/or information indicating the location of the medical care data (for example, a link to an image or the like), for example, in response to a request from the client terminal 12. Medical care data is images, reports, examination results, and other medical care processes acquired or created in medical examinations, tests, surgeries, and the like, or is data obtained as a result of medical care or information indicating the location of these (so-called links (aliases), etc.). The medical care support device 11 acquires medical care data to be used on the display screen from the server group 18.

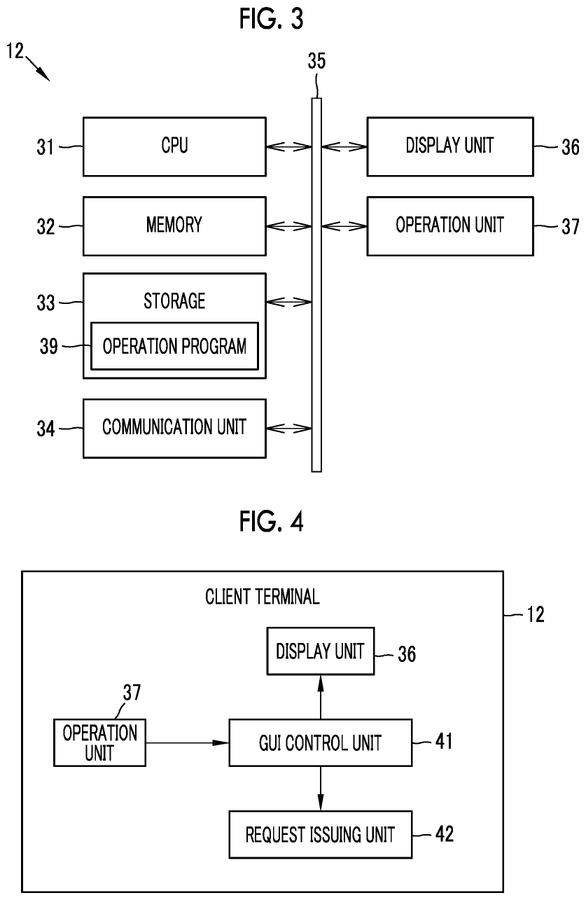

A display screen provided by the medical care support device 11 to the client terminal 12 refers to data used by the client terminal 12 to form a screen of a display unit 36 (see FIG. 3) of the client terminal 12. Further, the display screen provided by the medical care support device 11 to the client terminal 12 includes not only data for full-screen display that the client terminal 12 constitutes the display of the entire screen but also data that constitutes the display related to a part of the screen. For example, in the present embodiment, the medical care support device 11 provides the client terminal 12 with a display screen that can be displayed in a general window format on a part of the screen of the display unit 36.

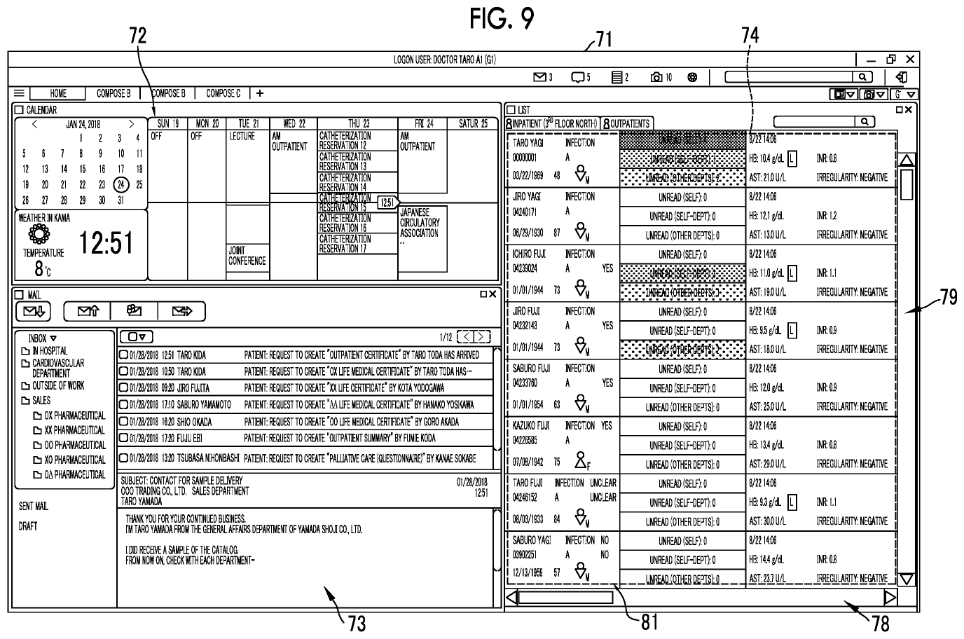

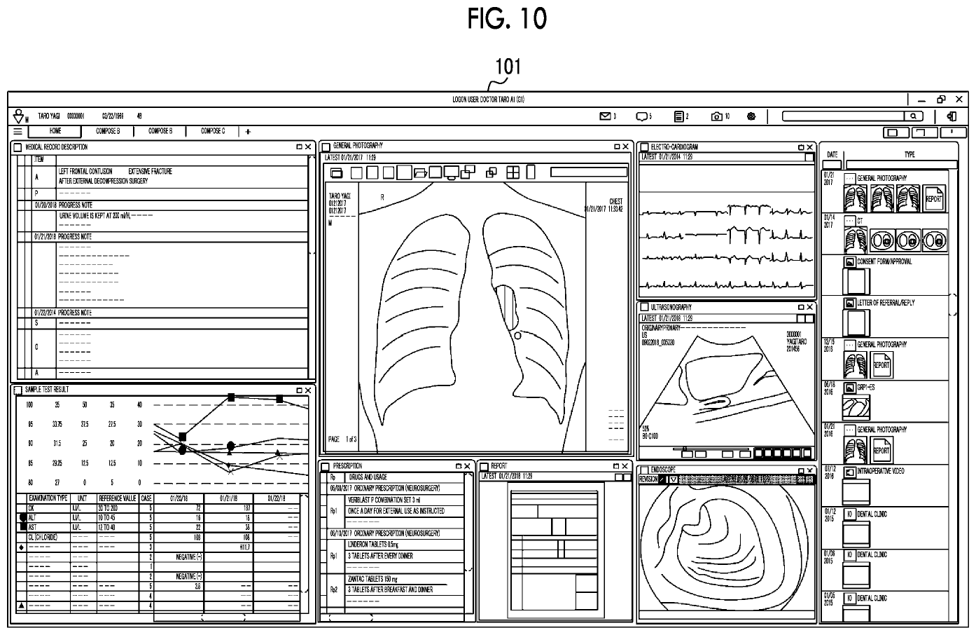

Specifically, the display screens provided by the medical care support device 11 to the client terminal 12 include an initial screen 71 (see FIG. 9), a clinical flow screen 81 (see FIG. 9), a timeline screen (not shown), a layout display screen 101 (see FIG. 10), and the like. The clinical flow screen 81 is a display screen for displaying patient identification information and a part or all of the medical care process in association with each other for each of a plurality of patients. Patient identification information is, for example, identification data (ID) such as the patient's name, date of birth, age, or gender, or a unique number and/or symbol given to the patient (hereinafter referred to as a patient ID). A medical care process refers to the process or result of medical care that has already been performed and that is scheduled to be performed in the future. Therefore, the medical care process may include not only medical care data that has already been acquired, but also medical care data that is scheduled to be acquired. The medical care data that is scheduled to be acquired is, for example, information regarding the presence or absence of an order for a specific examination, a scheduled date and time thereof, the type of medical care data that is scheduled to be acquired, and the like. The timeline screen is a display screen for displaying a part or all of the medical care process of a specific patient on one screen in a time series. The layout display screen 101 is a display screen for displaying a part or all of the medical care process of a specific patient by arranging them vertically and horizontally (for example, arranging them in a tile shape).

The medical care support device 11 provides a display screen to the client terminal 12 in a description format using a markup language such as extensible markup language (XML) data, for example. The client terminal 12 displays an XML format display screen using a web browser. The medical care support device 11 can provide a display screen to the client terminal 12 in another format such as JavaScript (registered trademark) object notation (JSON) instead of XML.

The server group 18 searches for medical care data in response to the request from the medical care support device 11, and provides the medical care data corresponding to the request to the medical care support device 11. The server group 18 includes an electronic medical record server 21, an image server 22, a report server 23, and the like.

The electronic medical record server 21 has a medical record database 21A for storing electronic medical records. An electronic medical record is a collection of one or a plurality of pieces of medical care data. Specifically, the electronic medical record includes, for example, medical care data such as a medical examination record, a result of a specimen test, a patient's vital sign, an order for examinations, a treatment record, or accounting data. The electronic medical record can be input and viewed using the client terminal 12.

A medical examination record is a record of the contents and results of the interview or palpation, the disease name, or the like. A specimen is blood or tissue collected from a patient, or the like, and a specimen test is a blood test, a biochemical test, or the like. A vital sign is data indicating a patient's condition such as a patient's pulse, blood pressure, or body temperature. An order for examinations is a request for examinations such as a specimen test, photography using various modality, report creation, treatment or surgery, medication, or the like. A treatment record is a record of treatment, surgery, medication, prescription, or the like. Accounting data is data related to consultation fees, drug fees, hospitalization fees, and the like.

The image server 22 is a so-called PACS (picture archiving and communication system) server, and has an image database 22A in which examination images are stored. An examination image is an image obtained by various image examinations such as computed tomography (CT) examination, magnetic resonance imaging (MRI) examination, X-ray examination, ultrasonography, and endoscopy. These examination images are recorded in a format conforming to, for example, the digital imaging and communications in medicine (DICOM) standard. The examination image can be viewed using the client terminal 12.

The report server 23 has a report database 23A for storing an interpretation report. An interpretation report (hereinafter simply referred to as a report) is a report that summarizes the interpretation results of the examination image obtained by the image examination. The interpretation of the examination image is performed by a radiologist. The report can be created and/or viewed using the client terminal 12.

A patient ID is attached to each of the above electronic medical records, examination images, and reports. In addition to the patient ID, information that identifies the medical staff who input the medical care data for each piece of medical care data is attached to the electronic medical record. In addition to the patient ID, information that identifies the medical staff (specifically, the laboratory technician) who performed the examination is attached to the examination image. Information that identifies the medical staff (specifically, the radiologist) that created the report is attached to the report. Information that identifies the medical staff is an ID such as the name of the medical staff or a unique number and/or symbol given to each medical staff (hereinafter referred to as a medical staff ID).

The medical care support device 11, the client terminal 12, the learning device 13, and the servers 21 to 23 constituting the server group 18 are configured by installing an operating system program and an application program such as a server program or a client program based on a computer such as a server computer, a personal computer, or a workstation. That is, the basic configurations of the medical care support device 11, the client terminal 12, the learning device 13, and the servers 21 to 23 constituting the server group 18 are the same, and a central processing unit (CPU), a memory, a storage, a communication unit, etc., and a connection circuit for connecting these are provided. The communication unit is a communication interface (modem, router, LAN interface board, or the like) for connecting to the network 14 or the network 16. The connection circuit is, for example, a motherboard that provides a system bus and/or a data bus and the like.

As shown in FIG. 3, the client terminal 12 comprises a display unit 36 and an operation unit 37 in addition to a CPU 31, a memory 32, a storage 33, a communication unit 34, and a connection circuit 35. The display unit 36 is, for example, a display using a liquid crystal display or the like, and has at least a screen for displaying a display screen provided by the medical care support device 11. The operation unit 37 is, for example, a pointing device such as a mouse and/or an input device such as a keyboard. The display unit 36 and the operation unit 37 can form a so-called touch panel.

The client terminal 12 stores an operation program 39 in addition to the operating system program and the like in the storage 33. The operation program 39 is an application program for receiving the function of the medical care support device 11 by using the client terminal 12. In the present embodiment, the operation program 39 is a web browser program. Here, the operation program 39 can be a dedicated application program for receiving the function of the medical care support device 11. The operation program 39 may include one or a plurality of gadget engines for controlling a part or all of the display screen provided by the medical care support device 11. A gadget engine is a subprogram that exhibits various functions by operating alongside a web browser or the like.

In a case where the operation program 39 is activated in the client terminal 12, as shown in FIG. 4, the CPU 31 of the client terminal 12 functions as a graphical user interface (GUI) control unit 41 and a request issuing unit 42 in cooperation with the memory 32.

The GUI control unit 41 displays the display screen provided by the medical care support device 11 on the web browser in the display unit 36. The GUI control unit 41 controls the client terminal 12 in response to an operation instruction input using the operation unit 37, such as a button click operation with a pointer.

The request issuing unit 42 issues various processing requests (hereinafter referred to as processing requests) to the medical care support device 11 in response to the operation instruction of the operation unit 37. The processing request issued by the request issuing unit 42 is, for example, a distribution request for the display screen, an edit request for the display screen, or the like. The request issuing unit 42 transmits the processing request to the medical care support device 11 via the communication unit 34 and the network 16.

The distribution request for the display screen is for requesting the medical care support device 11 to distribute a display screen having a specific configuration. For example, the distribution can be received by designating any one of the clinical flow screen 81, the layout display screen 101, and the like, depending on the distribution request for the display screen.

The edit request for the display screen is for requesting the medical care support device 11 to edit the contents of the medical care data and the like to be displayed on the display screen after receiving the distribution of the display screen having a specific configuration from the medical care support device 11. For example, in a case where the distribution of the clinical flow screen 81 is received, the edit request for the display screen is a request for designating or changing a list of patients to be displayed, designating or changing a display target period of the medical care process, designating or changing the medical care process to be displayed, or sorting the display contents.

The distribution request and/or edit request for the display screen includes information such as a medical staff ID and an address on the network of the client terminal 12. The medical staff ID is entered on a login screen (not shown) for the medical care support system 10 (or the medical care support device 11).

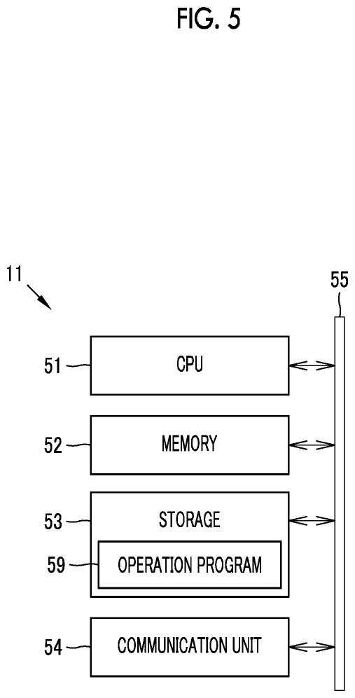

As shown in FIG. 5, the medical care support device 11 comprises a CPU 51, a memory 52, a storage 53, a communication unit 54, and a connection circuit 55. The medical care support device 11 can comprise a display unit and/or an operation unit as necessary, like the client terminal 12, and can be attached with a display unit and/or an operation unit as necessary, but in the present embodiment, the medical care support device 11 does not have a display unit and an operation unit.

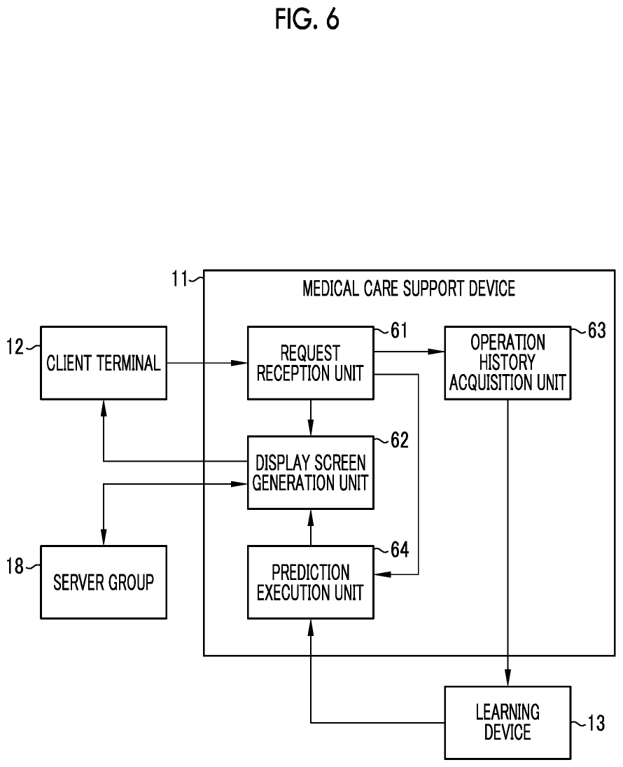

The medical care support device 11 stores an operation program 59 in addition to the operating system and the like in the storage 53. The operation program 59 is an application program for causing the computer constituting the medical care support device 11 to function as the medical care support device 11. In a case where the operation program 59 is activated, as shown in FIG. 6, the CPU 51 of the medical care support device 11 functions as a request reception unit 61, a display screen generation unit 62, an operation history acquisition unit 63, a prediction execution unit 64, and the like in cooperation with the memory 52.

The request reception unit 61 receives various processing requests such as a distribution request and an edit request for the display screen from the client terminal 12. In a case where the request reception unit 61 receives various processing requests, the request reception unit 61 inputs a processing instruction to each unit that executes the corresponding processing according to the content of the requested processing. For example, in a case where there is a distribution request for the display screen from the client terminal 12, the request reception unit 61 inputs a generation instruction of the corresponding display screen to the display screen generation unit 62. Similarly, in a case where there is an edit request for the display screen from the client terminal 12, the request reception unit 61 inputs an edit instruction of the corresponding display screen to the display screen generation unit 62. The request reception unit 61 also receives a request to log in to the medical care support device 11, and a login processing unit (not shown) executes login processing such as confirmation of the medical staff ID and password.

The display screen generation unit 62 generates or edits various display screens such as the clinical flow screen 81. The display screen generation unit 62 also functions as an operation proposal unit. In the present embodiment, in a case where there is a new distribution request for the display screen, the display screen generation unit 62 generates XML data representing the display screen, and in a case where there is an edit request for the display screen, the display screen generation unit 62 edits the XML data created earlier according to the request content.

The display screen generation unit 62 accesses the server group 18 as necessary, and acquires information regarding a medical care process or the like used for generating or editing the display screen. The display screen generation unit 62 can hold a part or all of the information regarding the medical care process or the like acquired from the server group 18 in order to reduce the access frequency to the server group 18. In a case where the login processing unit normally completes the login processing, the display screen generation unit 62 generates an initial screen 71 (see FIG. 9) to be displayed first after login. Further, in the case of creating or editing the initial screen 71, the display screen generation unit 62 acquires the information necessary for generating or editing the initial screen 71 from the server group 18, the client terminal 12, or another device or system that is linked with the medical care support system 10.

The operation history acquisition unit 63 extracts, for example, information related to the input operation of the client terminal 12 by the medical staff who is the user among various processing requests from the client terminal 12 received by the request reception unit 61 and acquires an operation history. User identification information for specifying a user who uses the client terminal 12 is attached to this operation history. The operation in which the operation history acquisition unit 63 extracts information related to the user's input operation of the client terminal 12 and acquires an operation history constitutes an operation history acquisition step.

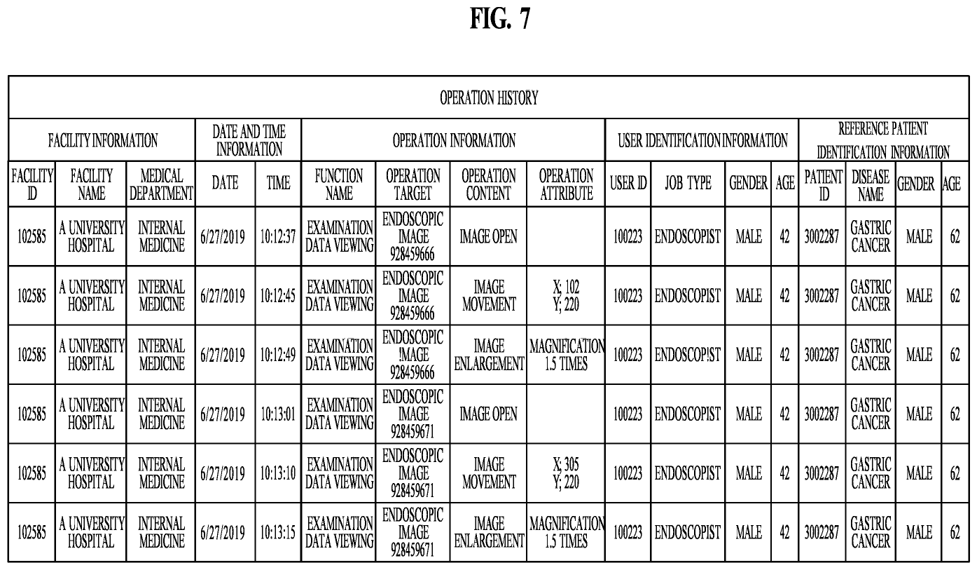

FIG. 7 shows an example of an operation history in a case where an input operation is performed on the client terminal 12, and for example, medical facility information, date and time information, operation information, user identification information, and reference patient identification information are included in the operation history. Further, the example shown in FIG. 10 is an example of an input operation in a case where the layout display screen 101 is displayed on the client terminal 12 and a case where an electronic medical record, an examination image, a report, or the like is edited.

The medical facility information is information about a medical facility in which the medical care support device 11 is installed, and includes information on a facility ID, a facility name, and a medical department, and the like. In addition, the present invention is not limited thereto, and the medical facility information may include the number of registered users, an address, contact information, and the like. For example, the medical facility information may be stored in advance in the storage 53 of the medical care support device 11, or may be acquired from the client terminal 12 or the server group 18.

The operation information is information related to an operation in a case where the medical staff who is a user inputs and operates the client terminal 12, and includes, for example, a function name, an operation target, an operation content, an operation attribute, and the like. Specifically, the function name includes examination data viewing, the operation target includes a file name of the endoscopic image, and the operation content includes instructions such as image OPEN of the endoscopic image (opening the endoscopic image file), image movement, and image enlargement. Furthermore, in a case where the operation content is image movement, the numerical value of the coordinates for the image movement is included as the operation attribute, and in a case where the operation content is image enlargement, the numerical value of the magnification ratio (display magnification) for the image enlargement is included as the operation attribute. In addition, the present invention is not limited thereto, in addition to endoscopic images, medical images such as X-ray images, examination results such as blood tests and pathological tests, and examination data such as examination reports may be used as operation targets, and the operation contents may include the order in which the operation targets are referred to, the change of the display layout input and operated by the user, and the like.

The user identification information attached to the operation history specifies the user who uses the client terminal 12, and includes a user ID, a job type, a gender, an age, and the like. The user ID is, for example, a number or the like entered in the case of logging in to the client terminal 12, and information such as a job type, a gender, and an age may be stored in advance in the storage 53 of the medical care support device 11 in association with the user ID, for example, or may be acquired from the client terminal 12 or the server group 18. The user identification information attached to the operation history may include personal information of the user (name of the medical staff who is the user, etc.), and in that case, as will be described later, in a case of transmitting the operation history to the external server, it is preferable to delete the portion of the user's personal information before transmitting. In the present embodiment, the user identification information does not include the user's personal information. Further, the user identification information may include years of experience and the like.

Further, the reference patient identification information attached to the operation history is patient identification information included in a display screen such as the layout display screen 101 displayed in a case where the client terminal 12 is used, that is, a patient ID associated with an electronic medical record edited by the client terminal 12, an examination image, and a report, or the like. Further, the disease name, gender, age, etc. other than the patient ID may be acquired from the client terminal 12 or the server group 18. In addition, the reference patient identification information may include the length of hospitalization and the like. The reference patient identification information attached to the operation history may include personal information of the patient (name of the patient, etc.), and in that case, as will be described later, in a case of transmitting the operation history to the external server, it is preferable to delete the portion of the patient's personal information before transmitting. In the present embodiment, the patient identification information does not include the patient's personal information.

As described above, the operation history acquisition unit 63 transmits the operation history with the user identification information and the like to the learning device 13 via the network 14. Like the medical care support device 11, the learning device 13 is a high-performance computer having a well-known hardware configuration such as the CPU 51, the memory 52, the storage 53, the communication unit 54, and the connection circuit 55, and a well-known operation system and the like installed therein, and further having a server function.

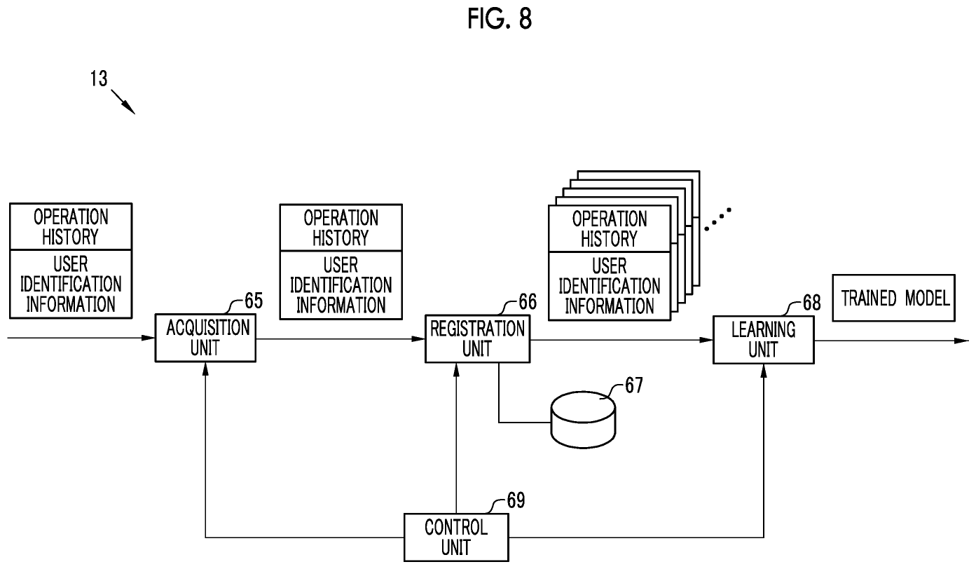

As shown in FIG. 8, the learning device 13 functions as an acquisition unit 65, a registration unit 66, a storage unit 67, a learning unit 68, and a control unit 69 by an operation system or the like. As described above, the acquisition unit 65 acquires the operation history transmitted from the medical care support devices 11 installed in the plurality of medical facilities A, B . . . X.

The control unit 69 controls the processing flow of the acquisition unit 65, the registration unit 66, and the learning unit 68. The registration unit 66 registers the operation history acquired by the acquisition unit 65 and the user identification information attached to the operation history in the storage unit 67. The storage unit 67 may be, for example, a part of the storage device provided in the learning device 13, or may be a storage device connected via the network 14.

The registration unit 66 registers an operation history as a sample for machine learning or the like by the learning unit 68. The registration unit 66 repeats the registration of the operation history from the medical care support device 11 while the medical care support system 10 is in operation.

The learning unit 68 performs machine learning for generating a trained model that outputs the next operation candidate in a case where any input operation is performed on the client terminal 12 by using a plurality of operation histories registered in the storage unit 67. In the present embodiment, the learning unit 68 specifically extracts the data as the operation target, the operation content (function) used as the input operation, or the order of the operation content used, and performs machine learning. The learning unit 68 reads the operation history registered in the storage unit 67 and the user identification information attached thereto, and generates a trained model from, for example, a plurality of operation histories having the same user ID or from a plurality of operation histories of users having the same attributes. Users having the same attributes refer to users having the same job type, medical department, patient's disease name, etc. included in the user identification information. Alternatively, in a case where a trained model is initially generated from the operation history of a user having the same attribute, and a predetermined number of operation histories having the same user ID are accumulated, a trained model may be generated from a plurality of operation histories having the same user ID. In a case where a trained model is generated from an operation history having the same user ID, it is possible to make a prediction optimized for each individual user, while in a case where a trained model is generated from an operation history of a user having the same attribute, there is an advantage that the operation history as a larger sample can be collected.

The learning device 13 transmits the trained model generated from the operation history to the medical care support device 11 via the network 14. In this case, the trained model is transmitted to the medical care support device 11 of the transmission source to which the operation history is transmitted by referring to the user ID attached to the operation history as a sample of the trained model.

The prediction execution unit 64 predicts the next operation in a case where the client terminal 12 is input and operated. The prediction execution unit 64 can be configured by using a trained model (so-called artificial intelligence (AI) program) generated by the learning device 13 described above.

The prediction execution unit 64 configured by using the trained model outputs the next operation candidate in a case where any input operation is performed on the client terminal 12. The operation of predicting the next operation candidate in a case where the prediction execution unit 64 inputs and operates the client terminal 12 constitutes a prediction execution step. The input operation of the client terminal 12 is acquired from the request reception unit 61 or the like, as in the case where the operation history acquisition unit 63 acquires the operation history. For example, in a case where a trained model is generated from the example of the operation history as shown in FIG. 7 described above and the prediction execution unit 64 is configured from this trained model, the prediction execution unit 64 focuses on the endoscopic image as the data which is an operation target. Then, in response to the input operation of the image OPEN of the endoscopic image, the next operation candidate of moving the endoscopic image is output as the next operation candidate. Alternatively, in response to the input operation of moving the endoscopic image, the next operation candidate of enlarging the endoscopic image is output. In addition, in the case of outputting the movement of the endoscopic image as the next operation candidate, it is preferable to output the endoscopic image with the movement amount attached thereto, and in the case of outputting the enlargement of the endoscopic image, it is preferable to output the endoscopic image with the magnification ratio attached thereto.

In the present embodiment, the display screen generation unit 62 makes a proposal to the client terminal 12 from the next operation candidate predicted by the prediction execution unit 64. Specifically, the display screen generation unit 62 generates or edits XML data representing the display screen by using the next operation candidate predicted by the prediction execution unit 64, and transmits the XML data to the client terminal 12. The operation in which the display screen generation unit 62 makes a proposal to the client terminal 12 from the next operation candidate predicted by the prediction execution unit 64 constitutes an operation proposal step.

The medical care support system 10 configured as described above operates as follows. First, in a case where the medical staff logs in to the medical care support system 10 using the client terminal 12, the display screen generation unit 62 generates the initial screen 71 shown in FIG. 9 based on the settings and the like set for each medical staff, and provides the initial screen 71 to the client terminal 12. Thereby, the client terminal 12 displays the initial screen 71 on the screen of the display unit 36.

The initial screen 71 has, for example, three display fields of a schedule display field 72, a mail display field 73, and a list display field 74. The display contents of the schedule display field 72 and the mail display field 73 are generated by a gadget engine, which is a part of the operation program 39 of the client terminal 12, by obtaining information from the client terminal 12 or other devices or systems. Further, in the present embodiment, the list display field 74 displays at least a part of the clinical flow screen 81. Therefore, the display screen generation unit 62 generates the initial screen 71 including the schedule display field 72 and the mail display field 73 that do not include the contents, and the list display field 74 that includes the contents of the clinical flow screen 81. The client terminal 12 uses a gadget engine to display the initial screen 71 supplemented with the contents of the schedule display field 72 and the mail display field 73 on the screen of the display unit 36.

In a case where all the contents to be displayed do not fit in the list display field 74, a scroll bar 78 and a scroll bar 79 for transitioning (so-called scrolling) the display contents of the list display field 74 are displayed in the list display field 74 or in the vicinity of the list display field 74. The scroll bar 78 is a GUI that is operated in a case where the display content of the list display field 74 is changed in the horizontal direction and a non-display portion is displayed. The scroll bar 79 is a GUI that is operated in a case where the display content of the list display field 74 is changed in the vertical direction and a non-display portion is displayed. The GUI control unit 41 performs such GUI display and control.

On the above initial screen 71, for example, in a case where a predetermined menu or the like is operated using a GUI such as a pointer (not shown), the request issuing unit 42 issues a distribution request for the display screen. In the present embodiment, in order to display the layout display screen 101 that is not displayed on the initial screen 71, an operation for displaying the layout display screen 101, for example, an input operation for selecting one of the patients displayed in the list display field 74 is executed by using the GUI. Thereby, the request issuing unit 42 issues a distribution request for the layout display screen 101.

In a case where the request issuing unit 42 issues a distribution request for the display screen, in the medical care support device 11, the request reception unit 61 receives the distribution request for the display screen, and the display screen generation unit 62 generates the display screen related to the distribution request for the display screen. In the present embodiment, the display screen generation unit 62 refers to the patient identification information (for example, the patient ID) included in the list display field 74, and acquires the information related to the patient. Specifically, an electronic medical record, an examination image, a report, and the like to which the same patient identification information as the patient identification information included in the list display field 74 is attached are appropriately acquired from the server group 18 or the like. Then, the layout display screen 101 is generated by using the information related to the patient acquired by referring to the patient identification information.

The GUI control unit 41 of the client terminal 12 receives the distribution of the display screen generated as described above, and the distributed screen is displayed on the screen of the display unit 36 instead of the initial screen 71, or is superimposed while leaving the initial screen 71 and displayed in another window or the like.

As described above, in a case where the display screen generation unit 62 generates the display screen related to the distribution request, before the generation of the display screen, at the same time as the generation of the display screen (in parallel with the generation of the display screen), or after the display screen is generated, the prediction execution unit 64 outputs the next operation candidate in response to the input operation. That is, the prediction execution unit 64 outputs the next operation candidate in response to the input operation of displaying the layout display screen 101 (see FIG. 10) on the client terminal 12. In a case where a trained model is generated from a plurality of operation histories including the example shown in FIG. 7, and the prediction execution unit 64 is configured from the trained model, the prediction execution unit 64 outputs the next operation candidate, for example, OPEN of the endoscopic image, in response to the input operation of displaying the layout display screen 101. Alternatively, in a case where the endoscopic image is included from the beginning (before the input operation) as the information for creating the layout display screen 101, in response to the input operation of image OPEN of the endoscopic image, the next operation candidate of moving the endoscopic image or enlarging the endoscopic image is output.

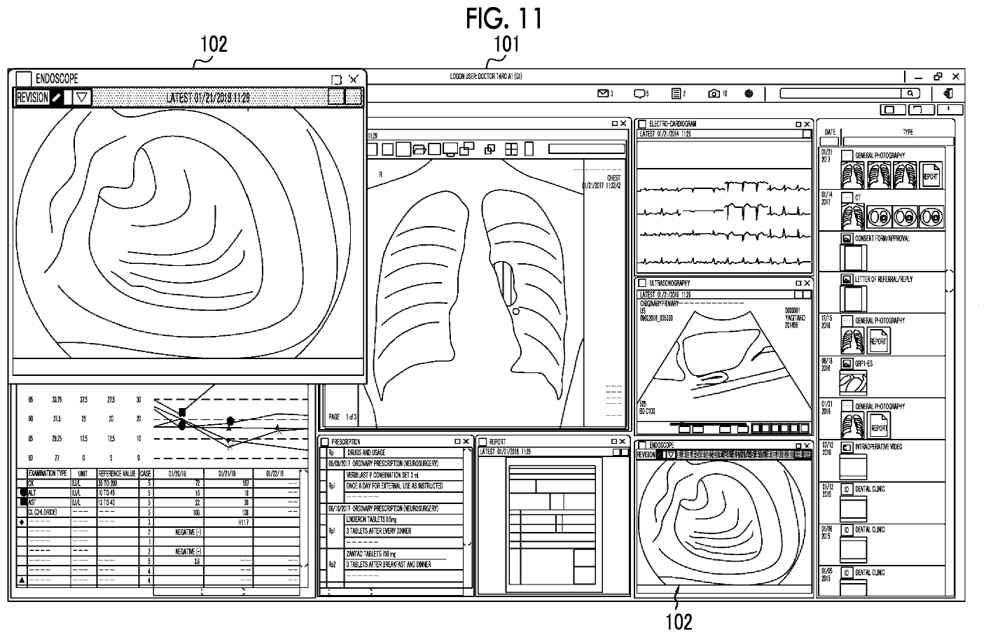

Next, the display screen generation unit 62 makes a proposal to the client terminal 12 based on the next operation candidate predicted by the prediction execution unit 64. That is, in response to the input operation of displaying the layout display screen 101 shown in FIG. 10, as shown in FIG. 11, the display screen in which the endoscopic image 102 is superimposed and displayed on the layout display screen 101 is edited. Here, the endoscopic image 102 superimposed and displayed on the layout display screen 101 is an endoscopic image to which the same patient identification information as the patient identification information acquired in the case of creating the layout display screen 101 is attached, for example, an endoscopic image with the latest image capture time. Alternatively, a computer-aided diagnosis (CAD) function or the like may be used to display an endoscopic image in which a portion suspected of having a disease is most clearly captured. In the case of endoscopic images, the parts that the user often refers to, such as the esophagogastric junction, duodenal bulb, anterior wall of the stomach, angular incisure, lower part of the body, middle part of the body, and upper part of the body, may be automatically laid out and displayed in the order in which the parts are often referred to.

In a case where the endoscopic image 102 is included from the beginning as the information for creating the layout display screen 101, instead of displaying the endoscopic image 102, the display screen in which the layout is changed, that is, the endoscopic image 102 is moved or the endoscopic image 102 is enlarged may be edited. Then, the display screen generation unit 62 distributes the edited display screen to the client terminal 12. Further, in this case, it is preferable that the prediction execution unit 64 predicts the movement amount and the magnification ratio of the endoscopic image 102, and the display screen generation unit 62 moves the endoscopic image 102 by the movement amount predicted by the prediction execution unit 64, and enlarges the endoscopic image 102 by the similarly predicted magnification ratio.

After that, the GUI control unit 41 of the client terminal 12 receives the distribution of the display screen edited as described above, and the distributed screen is displayed on the screen of the display unit 36 instead of the layout display screen 101 initially displayed.

As described above, since the operation history is transmitted to the learning device 13 as an external server to generate a trained model in the medical care support system 10 and the medical care support device 11 of the present embodiment, it is possible to collect a sufficient number of operation histories as a sample, and it is possible to improve the prediction accuracy of the trained model and the prediction execution unit 64. Further, in a case where the operation history is transmitted to the learning device 13 as an external server, a user ID or the like that does not include personal information is attached to the operation history as user identification information, and it is thus possible to avoid a risk of leakage of personal information.

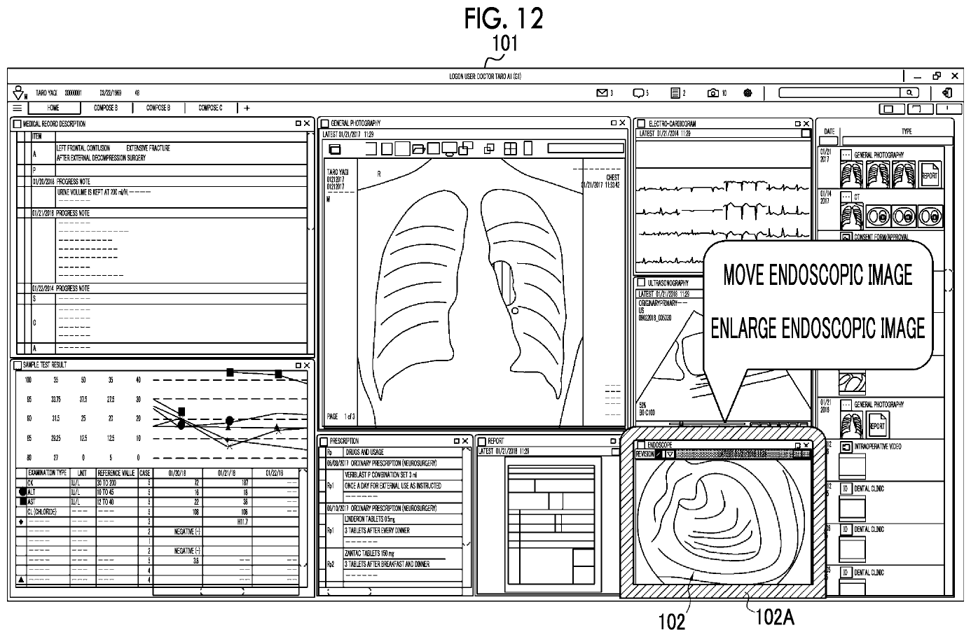

The editing of the display screen based on the next operation candidate predicted by the prediction execution unit 64, which is performed by the display screen generation unit 62, is not limited to the above operation, and for example, as shown in FIG. 12, the display of the endoscopic image 102 may be changed. In this case, the next operation candidate predicted by the prediction execution unit 64 is the image OPEN of the endoscopic image, but in a case where the endoscopic image 102 is included from the beginning as the information for creating the layout display screen 101, the display of the endoscopic image 102 may be changed.

In the example shown in FIG. 12, the frame line surrounding the endoscopic image 102 is thickened and the color of a frame line 102A is changed (for convenience of illustration, the inside of the frame line is shaded instead of changing the color). Then, similar to the above embodiment, the GUI control unit 41 of the client terminal 12 displays the edited layout display screen 101 on the screen of the display unit 36. Further, the present invention is not limited thereto, in a case where the endoscopic image 102 is included from the beginning as the information for creating the layout display screen 101, both the change of the display of the endoscopic image shown in FIG. 12 and the movement of the endoscopic image shown in FIG. 11 or the enlargement of the endoscopic image may be performed. Further, in FIG. 11, one endoscopic image 102 is displayed, but the present invention is not limited thereto, and a plurality of endoscopic images may be displayed.

Further, as another display based on the next operation candidate predicted by the prediction execution unit 64, which is performed by the display screen generation unit 62, the operation content (function) to be input and operated by the user may be displayed. For example, in a case where the next operation candidate predicted by the prediction execution unit 64 is the movement of the endoscopic image or the enlargement of the endoscopic image, the content may be displayed as the operation content 103 (see FIG. 12) to be input and operated by the user. Further, in a case where the order of the operation contents is learned as the trained model, focusing on the operation content input and operated last time by the user, the operation content to be input and operated next may be displayed.

### Second Embodiment

In the first embodiment, the learning unit 68 performs machine learning by extracting the data as the operation target in the operation history, the function used as the input operation, and the order of the input operations, but the content of machine learning from the operation history is not limited thereto, and in the second embodiment, the learning unit 68 may use the symptom, disease name, and examination name of the patient who has been treated by the user in the operation history as the operation target, and machine-learn what kind of examination data was referred to in the case of a predetermined symptom, disease name, and examination name. The configuration of the medical care support system 10 and the medical care support device 11 is the same as that of the first embodiment.

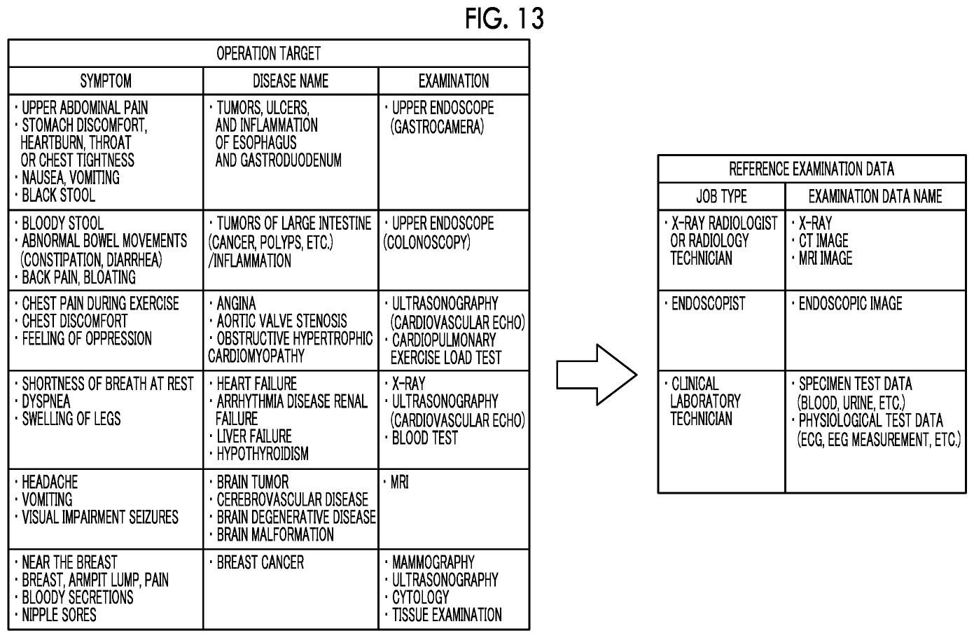

In the operation history shown in FIG. 13, the left side is a list of symptoms, disease names, and examinations of the patient as the operation target, and the right side is a list of examination data referred to by the medical staff who is the user in the case where the examination included in the operation target is performed. The referenced examination data differs depending on the job type of the user. Similar to the first embodiment, the medical care support device 11 of the present embodiment attaches the user identification information to the operation history shown in FIG. 13 and transmits the user identification information to the learning device 13.

In the present embodiment, the learning unit 68 of the learning device 13 uses the symptom, disease name, and examination name of the patient who has been treated by the user as an operation target, and machine-learns what kind of examination data was referred to in the case of a predetermined symptom, disease name, and examination name. Specifically, the learning unit 68 generates a trained model that outputs examination data names with high reference frequency for each job type of the user for a predetermined symptom, disease name, and examination name. The trained model generated by the learning unit 68 is transmitted to the medical care support device 11, and constitutes the prediction execution unit 64 of the medical care support device 11 as in the first embodiment.

In a case where the trained model is generated as described above, the medical care support system 10 operates as follows. Note that, the process is the same as in the first embodiment from the time when the medical staff logs in to the medical care support system 10 using the client terminal 12 until the layout display screen 101 is displayed. Then, the prediction execution unit 64 extracts the symptom, the disease name, and the examination name as the operation target from the data such as the electronic medical record, the examination image, and the report included in the layout display screen 101.

Then, the prediction execution unit 64 outputs the examination data name to be referred to next by the user from the extracted symptom, disease name, and examination name. For example, in a case where a trained model is generated from the example of the operation history as shown in FIG. 13 described above and the prediction execution unit 64 is configured from this trained model, focusing on the symptom, disease name, and examination name, when the user's job type is an endoscopist, the endoscopic image is predicted as the examination data name.

The display screen generation unit 62 makes a proposal to the client terminal 12 from the next operation candidate predicted by the prediction execution unit 64. That is, in response to the input operation of displaying the layout display screen 101, the display screen of the examination data (for example, the endoscopic image) to be referred to next is replaced with the layout display screen 101, or the display screen superimposed and displayed on the layout display screen 101 is edited. Then, the display screen generation unit 62 distributes the edited display screen to the client terminal 12. The GUI control unit 41 of the client terminal 12 receives the distribution of the display screen edited as described above, and the distributed screen is displayed on the screen of the display unit 36 instead of the layout display screen 101 initially displayed. By the above operation, it is possible to avoid the risk of leakage of personal information as in the first embodiment, and it is possible to improve the prediction accuracy of the trained model and the prediction execution unit 64.

### Third Embodiment

The example of the operation history that the learning unit 68 performs machine learning is not limited to the one shown in the first and second embodiments, and for example, the created document created by the user may be used as the operation target in the operation history, and a trained model may be created by extracting, from the operation history, the type and frequency of creation of the created document as the operation target, or in what order the created documents were created. The configuration of the medical care support system 10 and the medical care support device 11 is the same as that of the first embodiment.

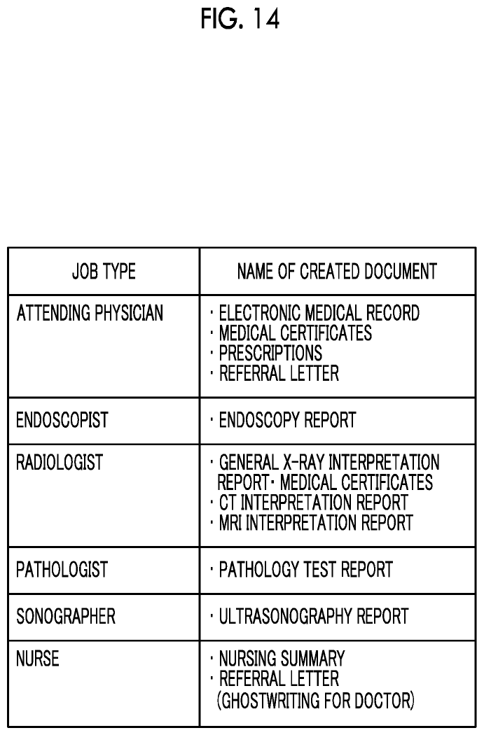

FIG. 14 is an example of the operation history used in the present embodiment, and in this operation history, the left column is the job type of the user, and the right column is the name of the created document created by the user corresponding to the left column. Similar to the first embodiment, the medical care support device 11 of the present embodiment attaches the user identification information to the operation history shown in FIG. 14 and transmits the user identification information to the learning device 13. In FIG. 14, a referral letter is described as the name of the created document created by the nurse, but this is a ghostwriter for the doctor and is a document that the doctor needs to finally confirm and sign. Similarly, medical certificates, prescriptions, etc. are also allowed to be created by staff other than doctors as assistants to doctors, provided that the doctor finally confirms and signs them, and they are ghostwritten by a staff member such as a nurse or a medical clerk, in some cases.

In the present embodiment, the learning unit 68 of the learning device 13 uses the name of the created document created by the user as an operation target, and machine-learns the type and frequency of creation of the created document, or the order in which the created document is created. Specifically, the learning unit 68 generates a trained model that outputs the name of the created document with high creation frequency for each job type of the user by machine learning. The trained model generated by the learning unit 68 is transmitted to the medical care support device 11 and used in the prediction execution unit 64 of the medical care support device 11 as in the first embodiment.

In a case where the trained model is generated as described above, the medical care support system 10 operates as follows. Note that, the process is the same as in the first embodiment from the time when the medical staff logs in to the medical care support system 10 using the client terminal 12 until the layout display screen 101 is displayed. Then, the prediction execution unit 64 extracts the job type of the user from various processing requests from the logged-in user ID. Then, the prediction execution unit 64 outputs the name of the created document that is likely to be created next by the user from the extracted job types of the user. For example, in a case where a trained model is generated from the example of the operation history as shown in FIG. 14 described above and the prediction execution unit 64 is configured from this trained model, focusing on the user's job type, when the user's job type is a radiologist, a general X-ray interpretation report is predicted as the name of the created document that is likely to be created next. In addition, in a case where the order in which the created document is created is learned for each job type of the user as a trained model, focusing on the document created last time by the user, the name of the created document that is likely to be created next may be predicted.

The display screen generation unit 62 makes a proposal to the client terminal 12 from the next operation candidate predicted by the prediction execution unit 64. That is, in response to the input operation that a user of a predetermined job type has logged in, a created document (for example, a general X-ray interpretation report) that is likely to be created next is set as the created document to be created next, and the display screen is replaced with the layout display screen 101, or the display screen superimposed and displayed on the layout display screen 101 is edited. Then, the display screen generation unit 62 distributes the edited display screen to the client terminal 12.

The GUI control unit 41 of the client terminal 12 receives the distribution of the display screen edited as described above, and the distributed screen is displayed on the screen of the display unit 36 instead of the layout display screen 101 initially displayed. In a case where the user has already created the document set by the display screen generation unit 62 as the created document to be created next, the created document may not be displayed. By the above operation, it is possible to avoid the risk of leakage of personal information as in the first embodiment, and it is possible to improve the prediction accuracy of the trained model and the prediction execution unit 64.

### Fourth Embodiment

In the third embodiment, the prediction execution unit 64 predicts the name of the created document that is likely to be created next for each job type of the user, but the prediction of the prediction execution unit 64 is not limited thereto, and a proposal may be made by predicting the name of the created document that is likely to be created according to the examination implementation status and the document creation status for each user or user's job type.

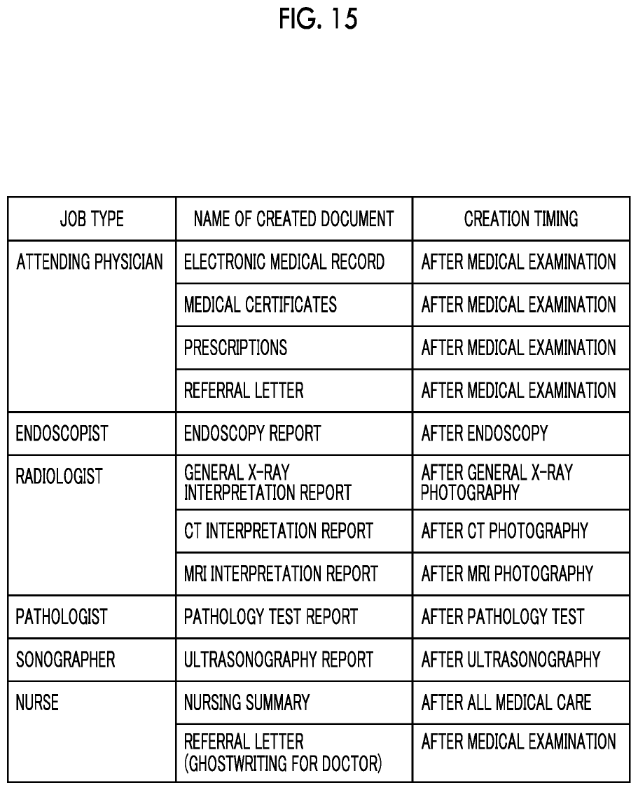

FIG. 15 is an example of the operation history used in the present embodiment, and in this operation history, the left column is the job type of the user, the center column is the name of the created document created by the user corresponding to the left column, and the right column is the creation time at which the user corresponding to the left column created the created document corresponding to the center column. As the creation time included in the operation history, a more detailed time or time slot may be acquired, or the time may be limited, for example, within several hours after the endoscopy, and a plurality of creation times may be acquired for one created document. Further, regarding the creation time, the time point or time slot at which the user created the created document may be acquired regardless of the medical care items such as after the examination and after the medical care.

In the present embodiment, the learning unit 68 of the learning device 13 uses the name of the created document created by the user as an operation target, and machine-learns the creation time of the created document. Specifically, the learning unit 68 generates a trained model that outputs the creation time at which the created document is frequently created for each job type of the user. The trained model generated by the learning unit 68 is transmitted to the medical care support device 11, and constitutes the prediction execution unit 64 of the medical care support device 11 as in the first embodiment.

In a case where the trained model is generated as described above, the medical care support system 10 operates as follows. The prediction execution unit 64 outputs a created document that is likely to be created and a creation time at which there is a high possibility of creating a created document for each job type of the user extracted from various processing requests. For example, in a case where a trained model is generated from the example of the operation history as shown in FIG. 15 described above and the prediction execution unit 64 is configured from this trained model, focusing on the user's job type, when the user's job type is a radiologist, a general X-ray interpretation report is predicted as the name of the created document that is likely to be created, and a time after general X-ray photography is predicted as the creation time that is likely to be created.

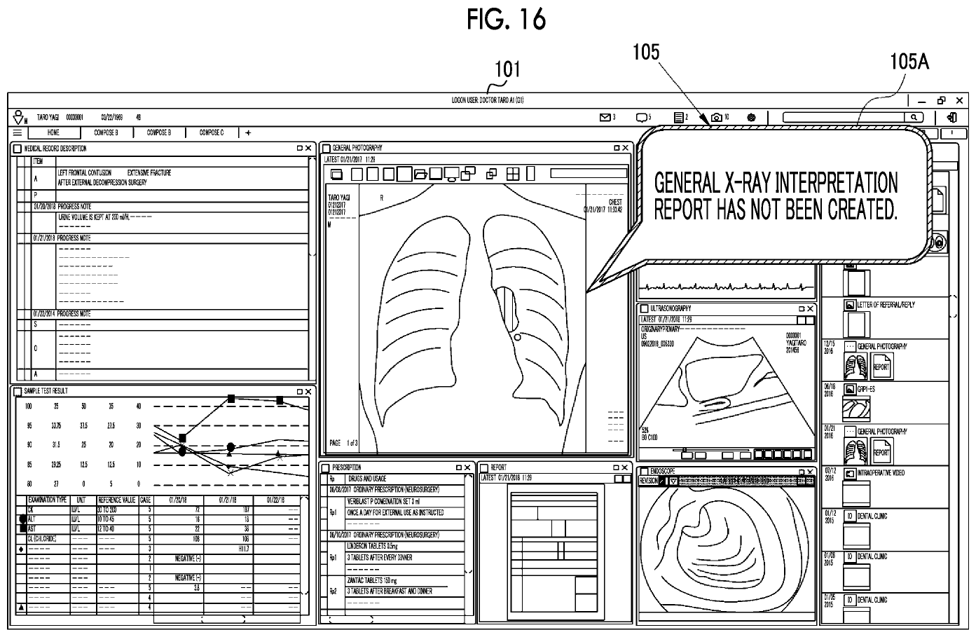

The display screen generation unit 62 makes a proposal to the client terminal 12 from the next operation candidate predicted by the prediction execution unit 64. In this case, the display screen generation unit 62 accesses the server group 18 after acquiring the prediction by the prediction execution unit 64, and also acquires the creation status of whether the predicted created document has been created or has not been created. As shown in FIG. 16, the display screen generation unit 62 sets a creation time that is likely to be created (for example, after general X-ray photography), which is predicted by the prediction execution unit 64, as the creation time at which the user should create the created document, and performs a display 105 prompting the creation of a created document to be created next (for example, a general X-ray interpretation report) at the creation time. In this case, the created document that is likely to be created next is set as the created document to be created next. In addition, the creation time referred to here is not limited to after any medical care, before medical care, etc., and is not limited to the time point such as hour and minute, but also includes the time slot such as morning and afternoon, the date, the day of the week, and the like. As the display 105 prompting the creation, a sentence “General X-ray interpretation report has not been created.” and a frame line 105A surrounding the sentence are thickened, and the color of the frame line 105A is different from the surrounding color. In this case, in a case where the user has already created the name of the created document to be created next, which is predicted by the prediction execution unit 64, the display 105 prompting the creation may not be performed.

Further, the proposal made by the display screen generation unit 62 may be made at a time later than the creation time predicted by the prediction execution unit 64, and for example, in a case where the created document to be created when a predetermined time has elapsed from the predicted creation time has not been created, the display 105 prompting the creation of the created document may be performed.

### Fifth Embodiment

In each of the above embodiments, as the contents to be machine-learned from the operation history, the machine learning is performed on the user-centered timing, such as the order of user's input operations, the frequency of creating created documents, the creation time at which created documents are created, and the creation status of created documents. However, the present invention is not limited thereto, and the creation time according to the medical care schedule of the patient in charge of the user may be machine-learned and predicted by the prediction execution unit 64. The configuration of the medical care support system 10 and the medical care support device 11 is the same as that of the first embodiment.

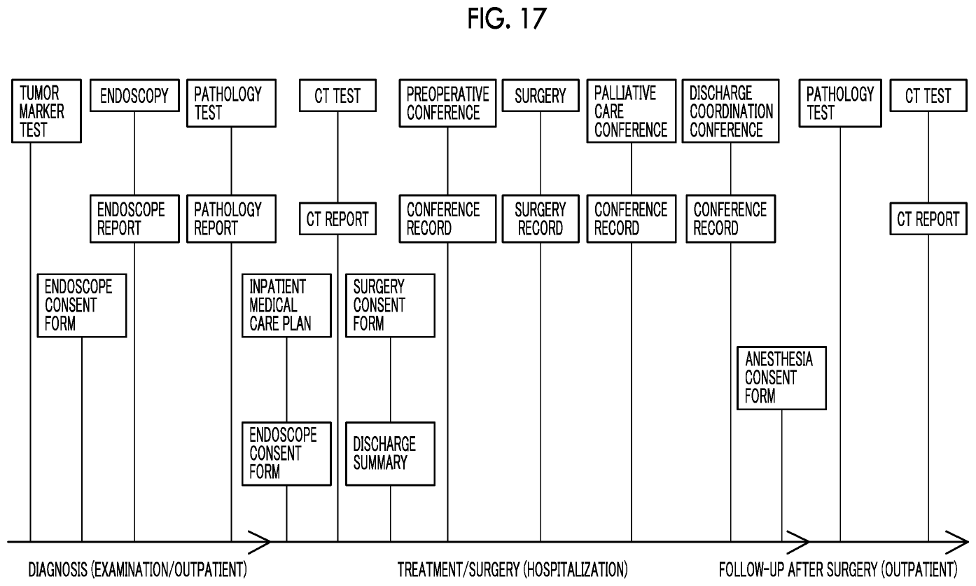

FIG. 17 is an example of the operation history used in the present embodiment, and the operation history arranges the medical care schedules of the patients in charge of the user in a time series, and is also called a so-called timeline. Further, the contents of this timeline may be created by the medical care support device 11 as a display screen and distributed to the client terminal 12, or the timeline may be edited by an input operation of the client terminal 12. The medical care items are listed at the top of the timeline, and the names of the created documents corresponding to the medical care items are listed below the medical care items. The time series shown at the bottom shows the period during which the patient is diagnosed before surgery, the period during which the patient is hospitalized for treatment and surgery, and the period during which the patient is followed up after surgery. Note that, FIG. 16 is an example of a patient with gastric cancer, and the job type of the user of the client terminal 12 is a surgeon.

In the present embodiment, the learning unit 68 of the learning device 13 uses the created document name corresponding to each medical care item for the medical care item of the patient in charge of the user as an operation target, and machine-learns the creation time according to the medical care schedule of the patient. That is, the learning unit 68 generates a trained model that outputs the creation time at which the created document is frequently created according to the medical care schedule of the patient. The trained model generated by the learning unit 68 is transmitted to the medical care support device 11, and constitutes the prediction execution unit 64 of the medical care support device 11 as in the first embodiment.

In a case where the trained model is generated as described above, the medical care support system 10 operates as follows. The prediction execution unit 64 outputs, for the medical care items of the patient in charge of the user extracted from various processing requests, the created document corresponding to each medical care item and the creation time at which the created document is frequently created in the medical care schedule. For example, in a case where a trained model is generated from the example of the operation history as shown in FIG. 16 described above and the prediction execution unit 64 is configured from this trained model, focusing on the medical care schedule of the patient in charge of the user, for example, in a case where the medical care item includes an endoscopy, an endoscopy consent form and an endoscope report are predicted as the name of the created document that is likely to be created, and a predetermined time before the endoscopy, a predetermined time after the endoscopy, or the like is predicted as the creation time that is likely to be created.

The display screen generation unit 62 makes a proposal to the client terminal 12 from the next operation candidate predicted by the prediction execution unit 64. In this case, the display screen generation unit 62 sets a creation time that is likely to be created, which is predicted by the prediction execution unit 64, (for example, after a predetermined time of endoscopy), as the creation time at which the user should create the created document, and edits the display screen of which the created document to be created next (for example, the endoscope report) is replaced with the display screen being displayed, or is superimposed and displayed on the display screen being displayed at the creation time. In this case, the created document that is likely to be created next is set as the created document to be created next. Then, the display screen generation unit 62 distributes the edited display screen to the client terminal 12.

The GUI control unit 41 of the client terminal 12 receives the distribution of the display screen edited as described above, and the distributed screen is displayed on the screen of the display unit 36. In a case where the user has already created the name of the created document to be created next, which is predicted by the prediction execution unit 64, the display of the created document may not be performed.

Alternatively, in a case where the prediction execution unit 64 predicts the creation time, a display prompting the creation may be performed as in the fourth embodiment. In addition, the creation time referred to here is not limited to after any medical care, before medical care, etc., and is not limited to the time point such as hour and minute, but also includes the time slot such as morning and afternoon, the date, the day of the week, and the like. As the display 105 prompting the creation, a sentence “General X-ray interpretation report has not been created.” and a frame line 105A surrounding the sentence are thickened, and the color of the frame line 105A is different from the surrounding color. In this case, in a case where the user has already created the name of the created document to be created next, which is predicted by the prediction execution unit 64, the display 105 prompting the creation may not be performed. By the above operation, it is possible to avoid the risk of leakage of personal information as in the first embodiment, and it is possible to improve the prediction accuracy of the trained model and the prediction execution unit 64.

In the fourth and fifth embodiments described above, machine learning is performed on the creation time at which the user created the created document, and the display of the created document or the display prompting the creation is performed at the creation time predicted by the prediction execution unit 64, but the present invention is not limited thereto. For example, after machine learning about the type of examination or treatment (including surgery or treatment) ordered by the user and the order time at which the user ordered the examination or treatment from the operation history of the user, the prediction execution unit 64 makes a prediction in the same manner as in each of the above embodiments. Then, from the prediction of the prediction execution unit 64, the display screen generation unit 62 may perform the display of the examination or treatment to be ordered or display the user to prompt the user to order the examination or treatment (for example, a sentence such as “MRI examination order has not been issued.” is displayed on the display screen.) at the order time at which the user should order. In addition, the order time referred to here is not limited to after any medical care, before medical care, etc., and is not limited to the time point such as hour and minute, but also includes the time slot such as morning and afternoon, the date, the day of the week, and the like. In this way, in the case of predicting the order time at which the user should order, in the operation history, the time point or time slot at which the user ordered may be acquired as the order time regardless of the medical care items such as after the examination and after the medical care. Thereby, it possible to learn the exact tendency of the user, such as, for example, ordering examinations at the time slot during the morning hours since it takes time to process pathological tests, or performing necessary examinations and document creation by then since the day of the week for surgery is determined by the medical facility.

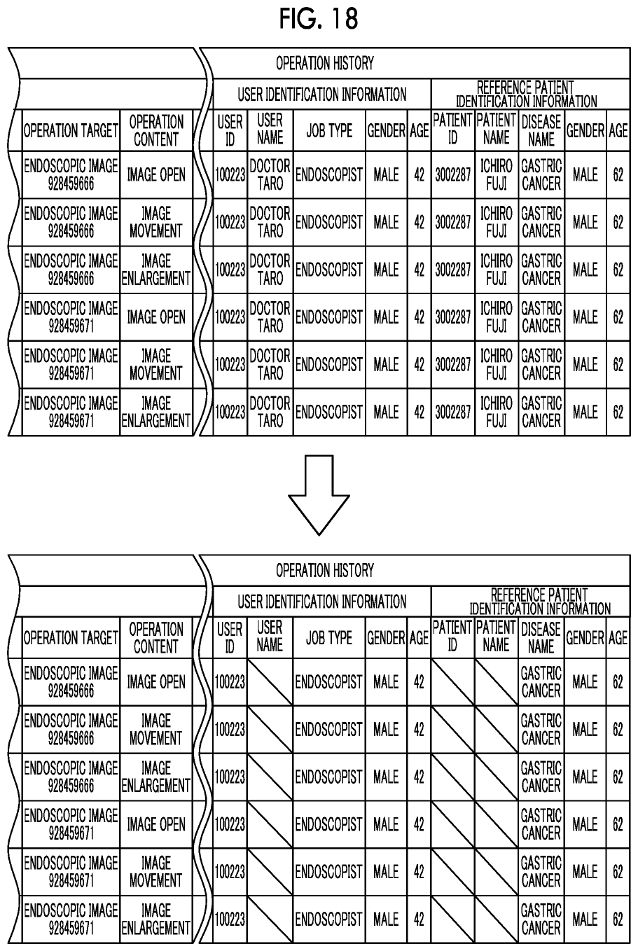

In each of the above embodiments, in a case where the operation history is transmitted to the learning device 13, a user ID or the like that does not include personal information is attached to the operation history. However, as shown in FIG. 18, in a case where the operation history acquired by the operation history acquisition unit 63 includes the personal information, or in a case where personal information of the patient in charge of the user is also included in the operation history, the portion of the personal information (user name, patient ID, patient name, etc.) may be deleted and then transmitted to the learning device 13. Thereby, it is possible to more reliably avoid the risk of leakage of personal information.

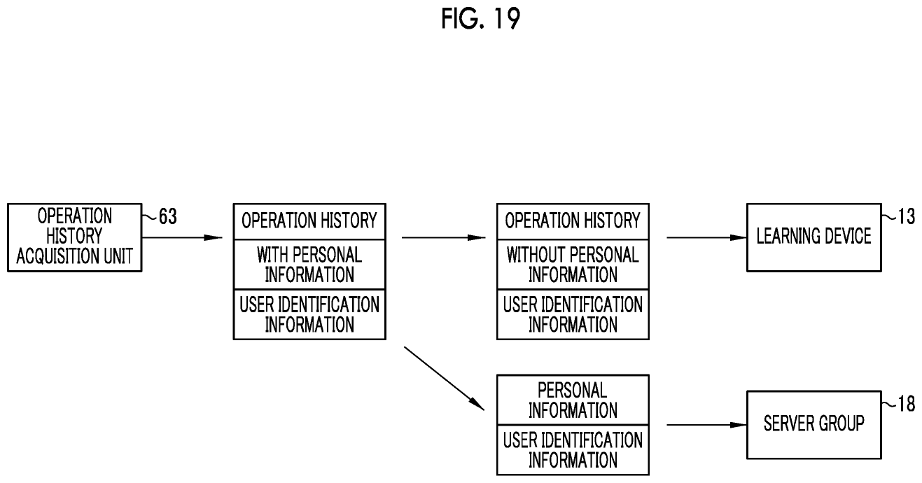

Further, as shown in FIG. 19, it is preferable that the portion of the deleted personal information is accumulated in the server group 18 installed in the same medical facility as the medical care support device 11, and the operation history in which the portion of the personal information is deleted is transmitted to the learning device 13. It is preferable to attach a user ID as user identification information corresponding to the operation history to the portion of the personal information deleted from the operation history. Thereby, in a case where the trained model generated by the learning device 13 is used in the prediction execution unit 64, the personal information accumulated in the server group 18 can be read out and the part related to the personal information can be restored. The server group 18 is an example of an internal storage device installed in the same medical facility as the client terminal.

In each of the above embodiments, hardware structures of the processing units that execute various processes such as the GUI control unit 41, the request issuing unit 42, the request reception unit 61, the display screen generation unit 62, the operation history acquisition unit 63, the prediction execution unit 64, the acquisition unit 65, the registration unit 66, the storage unit 67, the learning unit 68, and the control unit 69 are various processors as shown below. The various processors include a central processing unit (CPU) as a general-purpose processor functioning as various processing units by executing software (program), a programmable logic device (PLD) as a processor of which the circuit configuration can be changed after manufacturing such as a field programmable gate array (FPGA), a dedicated electrical circuit as a processor having a circuit configuration designed exclusively for executing various kinds of processing, and a graphical processing unit (GPU), and the like.

One processing unit may be configured by one of various processors, or may be configured by a combination of the same or different kinds of two or more processors (for example, a combination of a plurality of FPGAs, a combination of a CPU and an FPGA, or a combination of a GPU and a CPU). In addition, a plurality of processing units may be configured by one processor. As an example of configuring a plurality of processing units by one processor, first, as represented by a computer, such as a client or a server, there is a form in which one processor is configured by a combination of one or more CPUs and software and this processor functions as a plurality of processing units. Second, as represented by a system on chip (SoC) or the like, there is a form of using a processor for realizing the function of the entire system including a plurality of processing units with one integrated circuit (IC) chip. Thus, various processing units are configured by using one or more of the above-described various processors as hardware structures.

More specifically, the hardware structure of these various processors is an electrical circuit (circuitry) in the form of a combination of circuit elements, such as semiconductor elements. According to another aspect of the present invention, there is provided a medical care support device comprising a processor configured to acquire operation histories in a case where a plurality of terminal devices installed in a plurality of medical facilities are operated, from the terminal devices, predict next operation candidates in a case where the terminal devices are input and operated by using a trained model generated by learning the acquired operation histories by an external server installed outside the medical facilities, and make proposals to the terminal devices from the next operation candidates.

It goes without saying that the present invention is not limited to the above-described embodiment, and various configurations can be adopted as long as the gist of the present invention is not deviated. Further, the present invention is employed to a storage medium for storing the program in addition to the program.

From the above description, a medical care support device according to the following Additional Item 1 can be grasped.

[Additional Item 1]

A medical care support device comprising a processor, in which the processor is configured to acquire an operation history in a case where a user operates a terminal device installed in a medical facility, from the terminal device, predict a next operation candidate in a case where the terminal device is input and operated by using a trained model generated by an external server learning the acquired operation history, the external server being installed outside the medical facility, and make a proposal to the terminal device from the predicted next operation candidate.

### EXPLANATION OF REFERENCE

10: medical care support system

11: medical care support device

12: client terminal

13: learning device

14, 16: network

17: medical information system

18: server group

21: electronic medical record server

21A: medical record database

22: image server

22A: image database

23: report server

23A: report database

31, 51: central processing unit (CPU)

32, 52: memory

33, 53: storage

34, 54: communication unit

35, 55: connection circuit

36: display unit

37: operation unit

39, 59: operation program

41: graphical user interface (GUI) control unit

42: request issuing unit

61: request reception unit

62: display screen generation unit

63: operation history acquisition unit

64: prediction execution unit

65: acquisition unit

66: registration unit

67: storage unit

68: learning unit

69: control unit

71: initial screen

72: schedule display field

73: mail display field

74: list display field

78, 79 scroll bar

81: clinical flow screen

101: layout display screen

102: endoscopic image

102A, 105A: frame line

103: operation content

105: display

A1, A2, B1: doctor

G1, G2, G19: group

N1: technician

What is claimed is:

1. A medical care support device comprising:
a processor configured to:
acquire an operation history in a case where a user operates a terminal device installed in a medical facility, from the terminal device;
predict a next operation candidate in a case where the terminal device is input and operated by using a trained model generated by an external server learning the acquired operation history, the external server being installed outside the medical facility; and
make a proposal to the terminal device from the predicted next operation candidate.

2. The medical care support device according to claim 1, wherein user identification information for specifying the user who uses the terminal device is attached to the operation history.

3. The medical care support device according to claim 1,
wherein the trained model is generated by the external server learning the operation history, and
in a case where personal information is included in the operation history and the operation history is transmitted to the external server, the personal information is deleted.

4. The medical care support device according to claim 1,
wherein the trained model is generated by the external server learning the operation history, and
in a case where personal information is included in the operation history and the operation history is transmitted to the external server, the personal information is deleted, and a portion of the personal information in the operation history is accumulated in an internal storage device installed in the same medical facility as the terminal device.

5. The medical care support device according to claim 1, wherein the operation history includes at least examination data, as an operation target.

6. The medical care support device according to claim 1, wherein the operation history includes at least one of an order of examination data referred to by the user, a change of a display layout input and operated by the user, or a display magnification, as an operation target.

7. The medical care support device according to claim 1, wherein the operation history includes at least one of a type of created document created by the user, an order in which the created document is created, or a creation time of the created document, as an operation target.

8. The medical care support device according to claim 1, wherein the operation history includes at least one of a function used by the user or an order in which the function is used, as an operation target.

9. The medical care support device according to claim 1, wherein the operation history includes at least a type of examination or treatment ordered by the user through the terminal device.

10. The medical care support device according to claim 1, wherein the operation history includes at least an order time indicating a time point or a time slot at which the user ordered an examination or treatment through the terminal device.

11. The medical care support device according to claim 1, wherein the processor displays examination data on the terminal device as the proposal.

12. The medical care support device according to claim 1, wherein the processor changes a layout of examination data displayed on the terminal device as the proposal.

13. The medical care support device according to claim 1, wherein the processor performs, as the proposal, a display of a created document to be created by the user, or a display prompting creation of the created document by using the terminal device.

14. The medical care support device according to claim 1, wherein the processor displays, as the proposal, an operation content to be input and operated by the user by using the terminal device.

15. The medical care support device according to claim 1, wherein the processor performs, as the proposal, a display of an examination or treatment to be ordered by the user, or a display prompting the user to order the examination or treatment by using the terminal device.

16. The medical care support device according to claim 1, wherein the processor makes the proposal according to a time point or a time slot.

17. A medical care support system comprising the medical care support device, the terminal device, and the external server according to claim 1.

18. An operation method of a medical care support device, the operation method comprising:
acquiring an operation history in a case where a terminal device installed in a medical facility is operated, from the terminal device;
predicting a next operation candidate in a case where the terminal device is input and operated by using a trained model generated by an external server learning the acquired operation history, the external server being installed outside the medical facility; and
making a proposal to the terminal device from the predicted next operation candidate.

19. A non-transitory computer readable recording medium storing an operation program of a medical care support device, the operation program comprising:
acquiring an operation history in a case where a terminal device installed in a medical facility is operated, from the terminal device;
predicting a next operation candidate in a case where the terminal device is input and operated by using a trained model generated by an external server learning the acquired operation history, the external server being installed outside the medical facility; and
making a proposal to the terminal device from the predicted next operation candidate.
